(19) United States
(12) Patent Application Publication
(10) Pub. No.: US 2007/0083099 A1
(43) Pub. Date: Apr. 12, 2007

(54) Path related three dimensional medical imaging

(76) Inventors: Stephen W. Henderson, Mountain View, CA (US); Thilaka S. Sumanaweera, Los Altos, CA (US); Ismayil M. Guracar, Redwood City, CA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/241,603

(22) Filed: Sep. 29, 2005

(51) Int. Cl. A61B 5/05

(52) U.S. Cl. 600/407

(57) ABSTRACT

A path in a three dimensional structure is identified with a processor. The structure for determining the path may be identified by selection of a location on a one or two-dimensional image. The processor then extrapolates the structure of interest from the location and generates the path. Navigating along a path through a three dimensional space limits complication. For example, a simple input provides for navigation forward, backward or remaining stationary along the path. Navigation may be used to localize calculations, to define Doppler related scanning regions and orientations, or to determine the representation of the scanned volume data.

### BACKGROUND

The present embodiments relate to three-dimensional (3D) medical imaging. In particular, navigation is along a path for three-dimensional medical imaging.

3D ultrasound scanning of a volume has potential to increase the speed and effectiveness of blood flow analysis. A single transducer orientation might allow measurements to be quickly taken over a substantial segment of a vessel and associated branches. For two-dimensional (2D) imaging techniques, the transducer is repositioned manually for each cross-sectional scan of the vessel. However, to take advantage of these potential benefits, certain user interface obstacles must be overcome.

In 2D scanning modes, users typically indicate certain measurement locations. For example, a user places a cursor over a point of interest and activates a measurement. The estimation of blood flow velocity using pulsed-wave Doppler scanning techniques is performed at a user-selected location. However, in live 3D ultrasound scanning modes, users may find similar navigation very difficult through a volume. 3D navigation techniques, such as “fly through” or navigating within 2D cross-sections, may be demanding on the concentration and dexterity of the operator. This is especially true for an anatomical object, such as a blood vessel, which is typically narrow, has a complex shape, and might diverge into several branches. The difficulty is further exacerbated in the typical case where an operator manually positions the transducer and thus has only one hand and divided visual attention to manipulate user interface controls.

### BRIEF SUMMARY

By way of introduction, the preferred embodiments described below include methods, instructions and systems for navigating in three-dimensional medical imaging associated with a path. Navigating along a path through a three dimensional space limits complication. For example, a simple input provides for navigation forward, backward or stationary along the path. The path is defined by the user or automatically by a processor. The structure for determining the path may be identified by selection of a location on a one or two-dimensional image. The processor then extrapolates the structure of interest from the location and generates the path. In addition to navigation, the path may be used for calculations or to define Doppler related scanning regions or orientations. The different features described above may be used alone or in combinations.

In a first aspect, a method is provided for navigating during three-dimensional medical imaging associated with a path. The path is nonlinear. A user navigates along the path in a volume in response to user input.

The navigation may assist in additional measurements or localized ultrasound scanning. Measurements, such as localized Doppler or color flow obtained during live scanning, are guided by the navigation. Real-time cursor navigation through the volume in a more manageable method assists in taking measurements. The cursor may simply be shown moving along the path, but the perspective of the volume may not change. The measurements associated with the different cursor positions are performed. Changing the representation of the volume according to location on the path may be useful for real-time or for analyzing previously captured volume data.

In a second aspect, a computer readable storage medium has stored therein data representing instructions executable by a programmed processor for navigating during three-dimensional medical imaging associated with a path. The instructions are for navigating along the path in a volume in response to one dimensional user input, the path being nonlinear, and generating a three-dimensional representation of the volume, guiding localized scanning and/or performing measurements as a function of a location on the path, the location being responsive to the navigating.

In a third aspect, a method is provided for navigating in three-dimensional medical imaging associated with a path. User indication of a location on a one or two-dimensional image is received. The location is associated with the path, such as identifying a structure for which the path is to represent. The path is used to direct some additional measurements or localized ultrasound scanning.

In a fourth aspect, a computer readable storage medium has stored therein data representing instructions executable by a programmed processor for navigating in three-dimensional medical imaging associated with a path. The instructions are for generating a one or two-dimensional image, receiving user indication of a location on the one or two-dimensional image, the location associated with the path, and selecting a volume for three-dimensional imaging as a function of the location.

In a fifth aspect, a method is provided for navigating in three-dimensional medical imaging associated with a path. A processor determines a three-dimensional path in a vessel from medical imaging data. A three-dimensional representation of flow for a region of interest or from scanning is generated as a function of the three-dimensional path.

The present invention is defined by the following claims, and nothing in this section should be taken as a limitation on those claims. Further aspects, features and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the embodiments. Moreover, in the figures, like reference numerals designate corresponding parts throughout the different views.

FIG. 1 is a flow chart diagram of one embodiment of a method for navigating in three dimensional medical imaging associated with a path;

FIG. 2 is a graphical representation of one embodiment of a two dimensional image;

FIG. 3 is a graphical representation of one embodiment of a three dimensional representation with a path;

FIGS. 4 and 5 are graphical representations of scan patterns for determining a three dimensional flow vector; and

FIG. 6 is a block diagram of one embodiment of a system for navigating in three-dimensional medical imaging.

### DETAILED DESCRIPTION OF THE DRAWINGS AND PRESENTLY PREFERRED EMBODIMENTS

Navigation during three-dimensional medical imaging includes mapping a path, such as a blood vessel path, through 3D ultrasound scan volume. 1D user navigation along the 3D path through the scan volume may simplify navigation. For identifying the path of interest, the user selects the structure, such as the vessel, to be mapped in 3D by extending simple and well-established 2D user interface scheme such as 2D B/Doppler mixed mode. For example, the user selects a location on a two dimensional image associated with the structure of interest. Once the path is determined, automated ultrasound measurements use the path. A region-of-interest (ROI) or Doppler gate for 2D or 3D color-flow or Doppler scanning is positioned as a function of the path and estimated width of the structure. A scan plane and/or scanning angle for 2D or 3D color-flow or Doppler modes are oriented as a function of the location on the path. The path may be stored for later recall and diagnosis.

The path-based navigation may improve efficiency and manageability of navigating cursors (e.g. PW Doppler gate, region of interest, or view location) through a 3D volume. The 2D mode where the path's initial point is selected may provide experienced users with a familiar environment. This environment may provide an easy way to launch into 3D scanning modes for which they may have less experience, thus helping to facilitate their use of the 3D modes. The path provides a basis for defining a limited color flow region of interest to help avoid excessively slow frame rates expected with 2D and 3D B/Color mixed modes, and may help limit distracting display of excessive amounts of extraneous color information. The path may be clinically useful and/or may direct other automated measurements, such as for rapid detection of stenosis in carotid arteries.

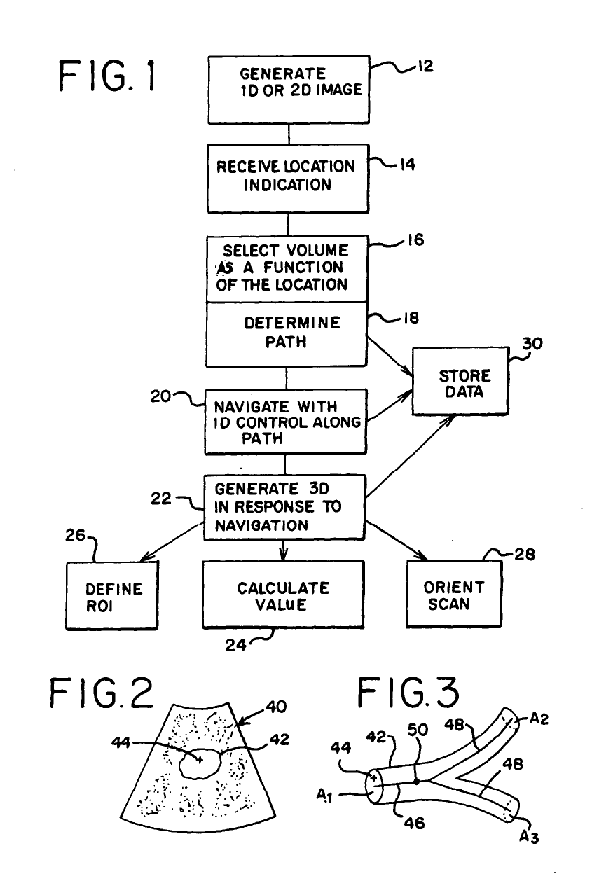

FIG. 1 shows a method for navigating in or during three-dimensional medical imaging associated with a path. The method is implemented with the system 60 of FIG. 6 or a different system. Additional, different or fewer acts may be provided. For example, the method is implemented with only acts 12 and 14, only acts 18 and 20, only acts 22 and 26 or only acts 22 and 28. As another example, acts 24-30 are optional. In one embodiment, the user is able to localize dynamically live scanning measurements, such as Doppler flow measurements. These measurements (Doppler or color flow) use a different form of localized scanning. The localization is controlled during live scanning by implementing acts 18 and 20 with one or more of the acts 26, 24, 28, or 22. Alternately, act 22 is a ‘special localized scanning as function of path.’ Such scanning, especially for Doppler, may not produce 3D volume data, but may produce time-varying 1D data.

In act 12, a one or two-dimensional image is generated. One and two-dimensional images include B-mode, flow mode, Doppler velocity, Doppler energy, M-mode, combinations thereof or other now known or later developed imaging modes. The one or two-dimensional image is generated as a function of one or two-dimensional scanning, respectively. For example, an M-mode image corresponds to repetitively scanning along a same scan line. As another example, a combination B- and flow mode image corresponds to linear, sector or Vector® scan formats over a plane or two-dimensional region. Fixed or electric focusing may be provided in elevation for a scan plane extending along azimuth and range dimensions.

FIG. 2 shows one embodiment of a two-dimensional image 40. The image includes a tissue structure 42, such as a vessel or chamber. In one embodiment, the image 40 is a B-mode image. In another embodiment, the image 40 is a gray scale B-mode image with color flow information provided within the structure 42. The image 40 represents the two dimensional scan region.

The one or two-dimensional scan is performed with a transducer. For example, a linear or curved linear transducer scans a patient. As another example, the one or two-dimensional scan is performed with a transducer operable to scan a volume. Wobbler or multiple dimensional arrays electronically or mechanically scan a volume or in three dimensions. For one or two-dimensional scanning with a volume capable transducer, the electrical and/or mechanical steering is controlled to scan a single image or repetitively scan a same scan plane relative to the transducer. Alternatively, a volume scan is performed and the one or two-dimensional image is generated by selecting data for a line or plane from the data representing the volume.

In act 14, a user indication 44 of a location on the one or two-dimensional image 40 is received. Any now known or later developed user navigation for the one or two-dimensional image is provided. The indication may be a location of a mouse or track ball controlled cursor, user touch or other user interface communication of a position on a screen for the image 40. The user places a cursor over or within the structure 42 of interest, such as a blood vessel, to indicate a point of interest. The user then indicates selection of the cursor location, such as by depressing a button or key. In alternative embodiments, the location is determined automatically, such as at a center, edge or other location associated with an automatically determined border or location of maximum flow.

The location of the user indication 44 of the structure 42 of interest selects a volume in act 16, such as a portion of the vessel, for three-dimensional imaging. The user indication 44 of act 14 may trigger a three dimensional scan. Alternatively, other user input triggers the three-dimensional scan. A volume around the tissue represented by the two dimensional image 40 and/or the structure 42 is scanned. For example, the two-dimensional image 40 corresponds to a plane on an edge, through the center or at another location relative to the volume. The volume scan is performed with a same or different transducer than the two or one-dimensional scans.

A path is determined in act 18 based on the location of the user indication 44. By scanning after receipt of the user indication 44, data representing the volume is acquired for positioning automatically or manually the path. The location identifies the structure 42, and the path is fit to the structure 42. FIG. 3 shows the structure 42 in three dimensions as a vessel with two branches. The user indication 44 is on a plane at the edge of the volume, but may be within the volume. The path 46 is determined as a center of the elongated structure 42, but may be at other locations within, on or adjacent to the structure 42. Since the vessel includes two branches, the path 46 includes two branches 48. Additional or no branches may be provided.

The path 46, including the branches 48, is three dimensional or nonlinear (i.e., a line that is not straight). The path 46 includes curves or angles along any of the dimensions. Alternatively, the path 46 extends along one or two dimensions, such as associated with a vessel that is parallel with a transducer face and does not curve or deviate from the parallel position through a length or portion of interest.

The path 46 is determined manually in one embodiment, such as the user tracing the path 46 using three-dimensional representations from different views or multiplanar reconstructions. Alternatively, the path 46 is manually traced in part, but with a processor fitting a line to manually selected points.

As yet another alternatively, the processor automatically determines the path 46 without further user input. The processor determines the path based on the user indication 44. Medical imaging data, such as the data representing the volume, is analyzed to determine the path. For example, after the cursor or user indication 44 is placed on a vessel 42, a system determines a path of the blood vessel 42 through a 3D scan volume.

A variety of mapping techniques are possible for determining the path of a vessel or structure 42 through a 3D scanning volume. Any now known or later developed path determination processes may be used, such as disclosed in U.S. Pat. Nos. 6,443,894 and 6,503,202, the disclosures of which are incorporated herein by reference. For boundary detection based processes, the path 46 is then determined from the detected boundary.

In one embodiment, the path 46 is determined by fitting a line through a contiguous region associated with lesser tissue reflection. A line is mapped or traced along a contiguous region associated with an absence of tissue reflection. B-mode or other tissue responsive imaging may have a reduced or no signal information for regions of fluid or flow, such as an interior of a vessel.

The user indication 44 defines an initial point ‘O₀’. The system considers B-mode reflection intensity over a grid of points lying within a spherical volume surrounding O₀. The grid corresponds to an equally spaced 3D grid or an acoustic grid. Full or sparse sampling of the data corresponding to the grid is provided. Other volume shapes, such as cubical or irregular, may be used. The size of the spherical volume is predetermined, set as a function of a detected border or may be adjustable by the operator. In one embodiment, the size is based on the application, such as providing a user adjustable size of 1 to 2 cm for vessel imaging. The medical data, such as B-mode reflected intensities, are used from a previous scan or are updated by a current scan of the spherical volume of interest or an entire volume.

Among the set of points lying within the sphere and associated data, the system determines points with intensities that fall below a predetermined, adaptive or user-determined threshold. Each point falling below the threshold defines a new candidate point ON representing a location with minimal or no tissue reflectivity. The system repeats the process for each new candidate point—defining a spherical volume around each new candidate point and considering the reflected intensities over grids of points lying within the spherical volumes. Only previously, unconsidered points are examined to see if they meet the threshold criterion. Previously considered points may be examined again, such as where scanning continues in real-time. The process repeats for each new candidate point from the subsequent spherical volumes until an edge of the scanned or entire volume is reached or no further candidate points are identified. Since the candidate points are limited to the different spherical volumes, the identified locations below the threshold may not include points and data associated with other structure. The process may complete having considered only a fraction of the total or entire 3D scanned volume.

The candidate points are then grouped by structure to identify points associated with the previously identified structure. Among the points meeting the threshold criterion, the system searches for the largest possible subset of candidate points that are spatially contiguous and contiguous with the initial point O₀. The identified points and not necessarily the associated data may be low pass filtered to remove noise from the identification. ‘Contiguity’ here means that a point is sufficiently close to at least one other point that is also in the contiguous subset. The distance criterion for contiguity is predetermined, adaptive or adjustable by the operator.

The system identifies all areas A_{N} where the maximum contiguous set intersects an edge of the scanning or scanned volume. The intersection of the three-dimensional structure with the edge of the volume generally or substantially defines an area. For each 2D area A_{N}, the system computes a center of mass C_{N}. A center of flow, offset from a center, an outer edge, inner edge, offset from the structure or other location may alternatively be identified.

For each center of mass C_{N}, the system initiates a process of curve fitting. Between two or more centers of mass C_{N}, the process fits a polynomial curve through all points contiguous to the centers of mass that lie within a certain distance D. A point the distance D inward from one center of mass is selected. The distance is predetermined, adaptive or configurable by the operator. The candidate points within a sphere or other volume centered at the center of mass is identified with a radius D are identified. A polynomial curve segment is fit to the identified candidate points. The curve extends from the center of mass to a point on the edge of the D radius sphere, the endpoint E_{N}. The system then defines a new sphere of distance D around the point E_{N}. All contiguous candidate points, excluding those previously used to fit the curve, are used to fit a new polynomial curve. As before, the endpoint of a curve segment is defined where the curve reaches surface of distance D from the starting point.

The process repeats for each area A_{N}. If at any juncture, any contiguous points overlap, a ‘branch’ in the vessel is identified. The continuous points currently being evaluated or having been evaluated in separate curve-fitting processes overlap based on the new portions of the D radius sphere. A centroid of the intersecting contiguous points is computed and from the last endpoint, curves are fitted from the nearest endpoints of each segment to meet at the overlap centroid point. Only the curve-fitting process that has already previously processed the overlapping points continues, while the one that is now ‘merging’ aborts. The process repeats until all points in the contiguous set have participated in curve fitting. The set of connected polynomial curve segments is the path 46 of a vein of interest and its branches. Other curve fitting may be used.

In another embodiment, the process described above is used to determine the path 46, but flow magnitude or energy is used instead of or in addition to tissue intensity. A line is fit through a contiguous region associated with greater flow magnitude. The threshold applied for identifying candidate points identifies locations with greater flow rather than lesser intensity.

In another embodiment, the path 46 is mapped as a function of the flow direction. A three dimensional flow vector within the structure is determined. A two dimensional transducer array allows interrogation of a same location from three or more different directions. For example, the flat or curved transducer face generally lies in the z and x dimensions. The position of the user indication 44 cursor is an initial or current ‘observed point’ ‘O’. The observed point may be determined as a location of greatest flow in a volume or area of contiguous flow with the user indicated point.

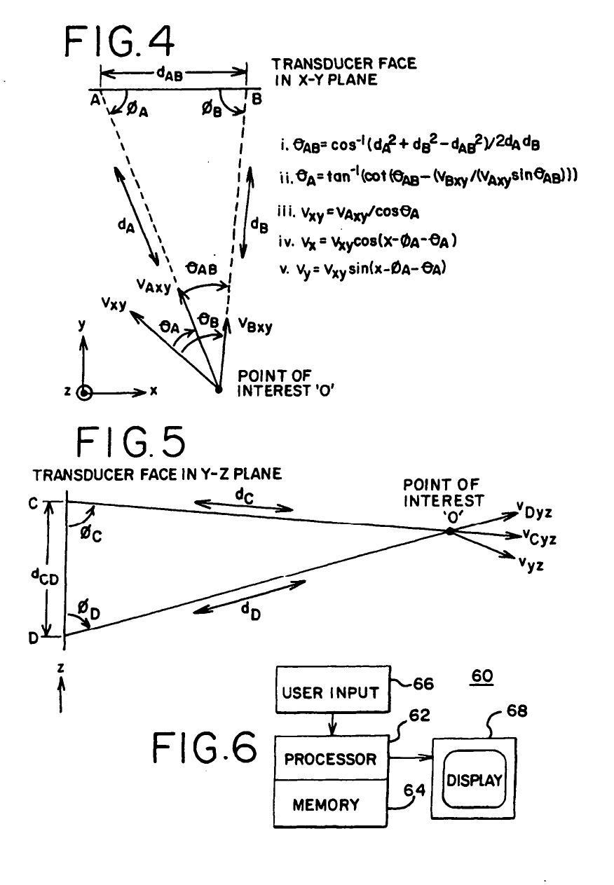

The ultrasound system measures Doppler frequency shifts, such as measuring with pulsed wave Doppler, at the observed point ‘O’ from two beam source locations ‘A’ and ‘B’ as shown in FIG. 4. ‘A’ and ‘B’ are located on the face of the transducer within a current scan plane, and separated by a predetermined, adaptive or user set distance D_{ab}. The distance is a great as possible given the transducer aperture and desired resolution. Flow velocities are measured along lines ‘AO’ and ‘BO’, and then expressions (i-v) are computed to determine projection of velocity vector in the current scan plane.

The ultrasound system then or simultaneously using coding measures Doppler frequency shifts at the observed point in a perpendicular plane. FIG. 5 shows beams emanating from points ‘C’ and ‘D’. Equations (i-v) determine the projection of velocity vector in the perpendicular plane. The 3D flow vector ‘V’ is calculated by combination of the velocity components determined for the two perpendicular planes.

Within the scanning volume, the system moves the observed point ‘O’ by small increment in the direction of the flow vector ‘V’. The increment may adapt as a function of the magnitude of the velocity vector, is preset or is user adjustable. The system determines the 3D flow vector for the new observed point. The process repeats to define the path 46 along the connected chain of observed points.

For identifying branches, the searches around each observed point for a direction of maximum flow near the next point O. If two or more diverging strong local maximums are found, the system spawns a separate tracing process for each direction. The system performs a directed search to find contiguous flow paths through the 3D scan volume. The path 46 corresponds to the path of the vessel and its branches. In yet another embodiment of determining the path in act 18, a line maps as a function of a medial axis transform of flow magnitude or velocity. Medial axis transform is a technique for determining the skeleton of structures in 3D. This method can be used on the volumes generated by using Doppler data, such as energy, velocity or variance. The Doppler data representing the 3D volume is input, and a set of doubly linked lists of points along the medial axis of the vessel is output. One doubly linked list corresponding to the axis of the vessel between nodes (bifurcations, etc.). Nodes are points connected to at least three doubly linked lists.

In other embodiments, different processes are used. Combinations of two or more of the processes may be used. Where different processes indicate different paths 46, the paths 46 are interpolated or averaged.

In act 20, the user navigates along the path 46 in the volume or structure 42 in response to one dimensional user input. After mapping the path 46 of the vessel, the operator navigates backwards and/or forwards along the path 46. The navigation may be stationary relative to the path, such as to allow rotation of a viewing direction from a same location. Using a simple user interface mechanism, user input indicates the direction of travel along the path 46. The user moves a cursor 50 or other indication of rendering position or region of interest from one end of the vessel to the other though the volume by employing one or more 1D navigation mechanisms. One control axis of a trackball or joystick controls movement forwards and backwards along the vessel. A dial or slider bar moves the location forwards or backwards along the path 46. Up/down or left/right selection of a 3-way self-centering toggle moves the location a fixed incremental displacement or at a fixed rate forwards or backwards along the vessel when the toggle is switched from its non-neutral position. The movement stops when the toggle returns to a neutral position. Two buttons provide forward and backward movement, respectively. In a voice activated system, voice commands such as “forward” or “back” cause the cursor 50 to move by a fixed displacement or rate along the path 46 of the vessel. Alternately, the user identifies numerical values or other labels at different positions along the path 46. Other mechanisms may be provided.

The one-dimensional navigation may be provided with additional control. For example, a trackball input of up and down moves navigates along the path and the input of left and right changes a size of a Doppler gate. The one-dimensional input is relative to the movement along the path provided by two-dimensional control of the movement and another parameter. One aspect of the user input maps to movement along the path, so is a one-dimensional navigation along the path. As another example, one-dimensional input provides navigation along the path 46. An additional input selects one branch from another.

In navigating along the path 46 through a vessel, branches 48 are selected. In response to user input, navigation in response to the one dimensional user input is along the selected branch 48. Any N-way selection technique, such as toggle switch, identifies or selects a branch 48 for continued navigation along the path 46. For example, upon reaching the point of a branch 48, the direction of a trackball or joystick is mapped to discrete angular sectors that each corresponds with a different vessel path 46. As another example, a toggle switch or dial is used to select the vessel path among a discrete set of choices. In the case of a voice-recognition enabled system, commands such as ‘right’, ‘left’, ‘center’, ‘center-left’, ‘first’, ‘second’, or others are mapped to the choice of blood vessel branches 48. As another example, the tree of branching vessels is navigated in a predetermined or logical order. No further inputs to select branches are used, instead along navigation sequentially along different branches based on forward or backward navigation along the path 46 off the branch 48. The branch order is defined according to any rule, such as branch direction with respect to the transducer face or relative sizes. The branches 48 map to different segments along the same 1D axis.

In optional act 22, a three-dimensional representation of the volume is generated as a function of the location 50 on the path 46. The transducer scans the volume for real time or later generating of an image after receiving the user indication 44. Surface, perspective, projection or any other now known or later developed rendering is used. For example, minimum, maximum or alpha blending is used to render from a viewers perspective at the location 50. The data used for rendering is the same or different data used to determine the path 46. For example, a different type of data from a same or interleaved time period is used. As another example, new data is acquired in a subsequent scan for imaging. A sequence of medical ultrasound images representing the volume in the patient is generated.

The data used for rendering corresponds to the flow data within the structure. Alternatively, tissue information representing the structure is rendered. In yet another embodiment, data from the entire scan region, including data outside of the structure 42, is used for rendering.

The rendering is responsive to the navigational control of the location 50. For example, a sequence of three-dimensional representations is rendered from a same data set viewed from different locations along the path 46 as the location is moved. Each time the location moves, another image is generated. The navigation may be provided in real-time, resulting in rendering in response to new scans and/or change in position of the location 50.

Many display representations are possible either singly or in combination with each other. Other representations of the volume in addition to or as an alternative of the rendered three-dimensional representations may be generated. Multiplanar reconstruction of orthogonal planes intersecting the location 50 may be generated. The system may display the 3D path 46 of the vessel as colored or heightened intensity points. The path 46 is shown in the midst of surrounding tissue within a representation using opacity or transparency rendering or with the path 46 displayed alone or in a similarly shaped containing volume or wire frame. The structure 42 or path 46 may be shown as a flattened projection into a projection plane chosen by the operator or determined automatically. The structure 42 may be displayed as an abstract linear profile of vessel thickness (e.g., a graph of thickness as a function of distance along the path 46). Different vessel branches may be related logically (e.g., with a connecting dotted line) to their parent vessel in the graphic display. Subsequent derived measurements could be plotted using these abstracted line segments as graph axes. The path 46 of the vessel may be presented as a straight-line segment projected in 3D space. The vessel's or structure's 42 estimated cross-sectional shape is modulated or graphed along this axis to produce an artificially straightened 3D view of the vessel. The logical relationships between a vessel and its diverging child branches are connected by dotted lines or otherwise interconnected rather than attempt to show their true spatial relationship. Other displays including or not including the path 46 may be used.

In optional act 24, a value is calculated as a function of the path 46. Measurements are guided manually or automatically by the path 46. For example, the ratio of maximum and minimum velocity along the path may be diagnostic. Characterization of flows at cross-sections through the path 46 may indicate diagnostic information. The cross-sectional area of the vessel or structure 42 perpendicular to the path 46 may indicate a constriction. Flow velocities at every point within the structure 42 identified as part of the path determination are calculated. The maximum flow magnitude throughout the whole vessel volume is identified. At the points of maximum flow magnitude, measurements of the ratio of maximum flow velocity to minimum flow velocity over the heart cycle for the same location may indicate the presence of stenosis. Localization of discontinuity in 3D velocity vectors may indicate blood turbulence. The total flow volume is measured by integrating flow velocity vectors across the vessel cross-section perpendicular to the path 46 at one or more locations. Different, fewer or additional measurements may be provided based on the path 46.

Once known, the path 46 of the vessel or structure 42 may be used to improve the efficiency and performance of 2D and 3D color flow and Doppler imaging. For example in optional act 26, a region of interest for flow imaging is defined as a function of the location 50 on the path 46. For 3D color flow mode, the region of interest (ROI) is a volume, such as a sphere, cube, 3D sector or other shape. The operator moves the ROI volume along the path 46 of the vessel in the navigation of act 20. The system automatically adjusts the position, dimensions and/or orientation of the ROI to the new location. For example, the dimensions and orientation are adjusted to encompass the estimated full width of the vessel or structure 42 at a user-selected location 50 along the path 46. Multiple pulses for flow imaging are transmitted to the ROI while minimizing the number of pulses to other regions, improving the scan frame rate as compared to a large ROI to cover the entire vessel. The amount of distracting extraneous color flow information presented to the operator is reduced, and the need for the operator to adjust manually the ROI dimensions is minimized.

In another embodiment of act 26, the region of interest is a Doppler gate or scanning focal position. The Doppler gate or focal position for real-time or continued scanning is set at the location identified by the navigation in act 20. The Doppler gate size and/or Doppler scanning angle may also be determined as a function of the path.

As another example of using the path 46 for flow imaging, a scan line or scan plane is oriented as a function of the location 50 on the path 46 in optional act 28. For 2D color flow or Doppler modes, the operator moves the 2D scan plane through the volume along the vessel path 46 by employing the navigation of act 20. At each location 50, the system automatically sets the angle of the scan plane to be transverse to the axis or path 46 or at a fixed angle relative to the path 46. In this way, a consistent and useful view of the vessel is continuously presented. If selected by the operator, the system may automatically orient the scan plane to be perpendicular to the transverse orientation and tangential to the path 46. Other angles may be used, such as adjusting the scanning angle to increase sensitivity to the blood flow. For example, a scanning angle of about 60 degrees relative to the direction of blood flow or the path 46 provides a better measurement of velocity than an angle that is nearly perpendicular to the flow. Such an automatic adjustment in scanning angle may also be used for 2D or 3D color flow and Doppler imaging. The scan line or scan plane may also be oriented for spectral Doppler imaging, such as positioning a Doppler gate at the location in response to the navigation of act 20.

In act 30, the path and any selected branches are stored. The scan data, navigation movements, regions of interest, non-selected branches, associated measurements from act 24 or other information may also or alternatively be stored. The storage allows subsequent display or indication of the stored path for analysis or diagnosis. Once the user traverses a particular path 46 through the vascular tree, such as by choosing one branch over the others at nodes, and generates the appropriate images, the path and the images may be stored in a disk or memory. At a later time, the path 46 is recalled and taken again by the same or different user, such as to confirm diagnosis. The path 46 can also be edited, augmented or subtracted. The derived measurement data can also be reviewed, edited, augmented or subtracted.

FIG. 6 shows one embodiment of a system 60 for navigating as a function of a path. The system 60 implements the method of FIG. 1 or other methods. The system 60 includes a processor 62, a memory 64, a user input 66 and a display 68. Additional, different or fewer components may be provided. For example, the system 60 is a medical diagnostic ultrasound imaging system that also includes a beamformer and a transducer for real-time acquisition and imaging. In another embodiment, the system 60 is a personal computer, workstation, PACS station or other arrangement at a same location or distributed over a network for real-time or post acquisition imaging.

The processor 62 is a control processor, general processor, digital signal processor, application specific integrated circuit, field programmable gate array, combinations thereof or other now known or later developed device for generating images, calculating values, receiving user input, controlling scanning parameters, storing data, recalling data, or combinations thereof. The processor 62 operates pursuant to instructions stored in the memory 64 or another memory. The processor 62 is programmed for navigating during three-dimensional medical imaging associated with a path.

The memory 64 is a computer readable storage media. The instructions for implementing the processes, methods and/or techniques discussed above are provided on the computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, filmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

The memory 62 may alternatively or additionally store medical data for generating images. The medical data is the scan data prior to navigation or image processing, but may alternatively or additionally include data at different stages of processing. For example, the medical data is image data for a yet to be or already generated three-dimensional representation.

The user input 66 is a keyboard, knobs, dials, sliders, switches, rocker switches, touch pad, touch screen, trackball, mouse, buttons, combinations thereof or other now known or later developed user input device. The user input 66 includes devices for implementing different functions in a common layout, but independent or separate devices may be used.

The display 68 is a CRT, LCD, projector, plasma, or other display for displaying one or two dimensional images, three-dimensional representations, graphics for the path, regions of interest or other information.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for navigating during three-dimensional medical imaging associated with a path, the method comprising:
navigating along the path in a volume in response to user input, the path being nonlinear; and
performing an additional localized scanning or measurement as a function of a location on the path, the location being responsive to the navigating.

2. The method of claim 1 further comprising generating a three-dimensional representation as a function of the location on the path.

3. The method of claim 1 wherein navigating comprises navigating along the path through a vessel as a function of one-dimensional user input.

4. The method of claim 1 wherein navigating comprises navigating in response to one-dimensional user input of forward, backward or stationary inputs.

5. The method of claim 1 wherein the path comprises at least first and second branches, further comprising:
selecting between the first and second branches in response to user input;
wherein navigating comprises navigating in response to the user input along the selected first or second branch, the user input being one-dimensional with respect to movement.

6. The method of claim 1 further comprising:
determining the path with a processor from medical imaging data.

7. The method of claim 6 wherein determining the path comprises:
fitting a line through a contiguous region associated with lesser tissue reflection;
fitting the line through the contiguous region associated with greater flow magnitude;
mapping the line as a function of flow direction;
mapping the line as a function of a medial axis transform of flow magnitude or velocity; or
combinations thereof.

8. The method of claim 6 wherein determining the path comprises receiving user indication of a location on a one or two-dimensional image, the location associated with the path.

9. The method of claim 8 wherein the one or two-dimensional image is generated as a function of one or two-dimensional scanning, respectively, with a transducer operable to scan a volume and wherein the three-dimensional representation is generated as a function of volume scanning with the transducer after receiving the user indication.

10. The method of claim 1 wherein performing the additional localized scanning or measurement as the function of the location on the path comprises calculating a value as a function of the location.

11. The method of claim 1 further comprising:
defining a region of interest as a function of the location on the path.

12. The method of claim 1 wherein performing the additional localized scanning or measurement as the function of the location on the path comprises orienting a scan line or scan plane as a function of the location on the path.

13. The method of claim 6 further comprising:
storing the path and any selected branches; and
subsequently indicating the stored path.

14. In a computer readable storage medium having stored therein data representing instructions executable by a programmed processor for navigating during three-dimensional medical imaging associated with a path, the storage medium comprising instructions for:
navigating along the path in a volume in response to one dimensional user input, the path being nonlinear and the one dimensional user input being with respect to movement; and
generating a three-dimensional representation, guiding localized scanning, measuring or combinations thereof as a function of a location on the path, the location being responsive to the navigating.

15. The instructions of claim 14 wherein generating the three-dimensional representation comprises generating a sequence of medical ultrasound images representing a volume in a patient, the sequence responsive to the navigation along the path.

16. The instructions of claim 14 wherein navigating comprises navigating in response to user input of forward, backward or stationary inputs.

17. The instructions of claim 14 wherein guiding localized scanning comprises steering as a function of the location.

18. The instructions of claim 14 wherein measuring comprises calculating a value as a function of the location.

19. A method for navigating in three-dimensional medical imaging associated with a path, the method comprising:
generating a one or two-dimensional image;
receiving user indication of a location on the one or two-dimensional image, the location associated with the path; and
selecting a volume as a function of the location.

20. The method of claim 19 wherein selecting the volume comprises determining the path with a processor from medical imaging data as a function of the location.

21. The method of claim 19 wherein the one or two-dimensional image is generated as a function of one or two-dimensional scanning, respectively, with a transducer operable to scan a volume; and
further comprising generating a three-dimensional representation of the volume as a function of volume scanning with the transducer after receiving the user indication.

22. The method of claim 20 further comprising:
navigating along the path in response to one dimensional user input, the path being nonlinear; and
generating a three-dimensional representation of the volume, calculating a value or guiding scanning as a function of a location on the path, the location being responsive to the navigating.

23. The method of claim 20 further comprising:
storing the path and any selected branches; and
subsequently indicating the stored path.

24. In a computer readable storage medium having stored therein data representing instructions executable by a programmed processor for navigating in three-dimensional medical imaging associated with a path, the storage medium comprising instructions for:
generating a one or two-dimensional image;
receiving user indication of a location on the one or two-dimensional image, the location associated with the path; and
selecting a volume as a function of the location.

25. The instructions of claim 24 wherein selecting the volume comprises determining the path with a processor from medical imaging data as a function of the location.

26. The instructions of claim 24 wherein the one or two-dimensional image is generated as a function of one or two-dimensional scanning, respectively, with a transducer operable to scan a volume; and
further comprising generating a three-dimensional representation of the volume as a function of volume scanning with the transducer after receiving the user indication.

27. A method for navigating in three-dimensional medical imaging associated with a path, the method comprising:
determining a three-dimensional path in a vessel with a processor from medical imaging data; and
scanning as a function of the three-dimensional path.

28. The method of claim 27 further comprising generating a three-dimensional representation of flow from scanning at an angle, the angle being a function of the three-dimensional path.

29. The method of claim 27 wherein scanning comprises positioning a Doppler gate as a function of the three-dimensional path.
